(19) United States
(12) Patent Application Publication
(10) Pub. No.: US 2003/0032669 A1
(43) Pub. Date: Feb. 13, 2003

(54) Pesticidal compositions and their use as protecting agents

(76) Inventors: Luc Rosalia Michael Verbruggen, Kasterlee (BE); Johan Maria Jozef Gors, Beerse (BE)

Correspondence Address: Philip S Johnson, Johnson & Johnson, One Johnson & Johnson Plaza, New Brunswick, NJ, 08933-7003, US

(21) Appl. No.: 10/182,934

(22) PCT Filed: Feb. 27, 2001
(86) PCT No.: PCT/EP01/02227

(30) Foreign Application Priority Data
Mar. 7, 2000 (EP) 00200807.6

(51) Int. Cl. A61K 31/27; A01N 47/10

(52) U.S. Cl. 514/479

(57) ABSTRACT

Compositions comprising one or more carbamic acid derivatives, in particular fenoxycarb; and one or more 4-(haloalkyl)phenoxyphenylureas, in particular flufenoxuron, in respective proportions such as to provide a synergistic effect against insects and/or acari. These compositions are useful for protecting any living or non-living material, such as crops, plants, fruits, seeds, building parts, biodegradable material and textiles against deterioration due to the action of pests and for treating animals.

The present invention relates to pesticide compositions and particularly to compositions which are able to provide an improved protecting effect against invertebrates such as insects and acari. More particularly, the present invention relates to pesticide compositions comprising specific carbamic acid derivatives, such as fenoxycarb, together with specific halobenzoylureas such as flufenoxuron, for protecting any living or non-living material, such as crops, plants, fruits, seeds, building parts made of wood, thatch or the like, biodegradable material and textiles against deterioration due to the action of pests such as insects and for treating animals, especially mammals, gallinaceae and anatidae, against such harmful invertebrates. This invention thus relates to the field of protection of plants, crops, warm-blooded animals, food, building materials and textiles through the control of pests.

Various classes of chemical compounds, for instance phenyl carbamates from GB-A-1,220,256 are already known as pesticides and in particular as insecticides. A class of carbamic acid derivatives, in particular substituted phenoxyethylcarbamic acid esters, are known from U.S. Pat. No. 4,215,139 as active ingredients in pesticide compositions for the control of invertebrates, particularly arthropods, nematodes and insects. These carbamic acid derivatives are insect growth regulators (hereinafter referred as “IGRs”) with juvenile hormone activity, also named juvenoids, which interfere with the hormonal system of pests and therefore produce deleterious effects on reproduction associated with twisted or curly wings. Other classes of IGRs include chitin synthesis inhibitors, which disrupt moulting in immature insects. Examples of chitin synthesis inhibitors include specific halobenzoylureas such as disclosed in U.S. Pat. No. 3,748,356 (including diflubenzuron) and EP-B-161,019 (including flufenoxuron). All of these compounds are IGRs with contact and/or stomach action which are used to kill or sterilize insects. IGRs affect insects mainly by disturbing normal activity of insect endocrine system and by causing premature death from abnormal moulting and metamorphosis. Hormones produced by the insect endocrine glands, i.e. brain hormone, ecdysone and juvenile hormone, are involved in the process that initiate and regulate moulting and metamorphosis, i.e. by which larvae form into adults.

More specifically, carbamic acid derivatives such as fenoxycarb and pyriproxyfen bind to juvenile hormone receptors and mimic their action by interfering with the moulting of early instar larvae, by inhibiting the metamorphosis to the adult stage and by affecting reproduction. Juvenile hormone mimics are especially effective to combat pest species wherein only adults, not larvae, are perceived as harmful or a nuisance (e.g. mosquitoes, flies, fleas), and also where large populations and not a low number of individual insects are harmful and for combating sedentary or low mobility insect targets that are not likely to quickly rise again because of re-infestation (e.g. scale insects, mealybugs). They are further useful in special instances where the safety of the compound and the lack of other control methods are the main considerations (e.g. pharaoh ant, Monomorium pharaonis). They are most useful in closed and protected environments and to control insects having a short lifetime cycle.

Halobenzoylureas such as diflubenzuron belong to a different class of IGRs producing its pesticide effect by disrupting the synthesis of chitin during moult. Its mechanism of action is by interaction with chitin, the major component of integument (the rigid exoskeleton covering insects), at any stage of the discontinuous development of insects when they shed their integument and create a new one larger in size. Flufenoxuron is another such chitin synthesis inhibitor, affecting thereby the integrity of the insect exoskeleton. Exposure of nymphs to such compounds causes improper attachment of the new cuticle during moulting and produces a cuticle that lacks some of the layers that would normally occur. Treated larvae either are incapable of emerging from the eggs or will therefore die at the next moult or during the ensuing instar because of rupture of the new malformed cuticle or from starvation. Treated adults lay non-viable eggs. Diflubenzuron and flufenoxuron are not juvenile hormone analogues and are not limited to the uses mentioned for such juvenoids. For instance, besides being an insecticide, flufenoxuron is also able to kill other kinds of arthropods (especially those belonging to subphylum Chelicerata), such as arachnids, in particular acari and mites (Pedigo L. P (1989), Entomology and pest management, Prentice Hall Inc., New Jersey).

The quantitative efficiency of juvenoids and chitin synthesis inhibitors is already well documented for a number of insects. For instance U.S. Pat. No. 4,215,139 discloses that 100% reduction of population of adult grain weevils Sitophilus granarius is achieved after 50 days by treating wheat with 1 μg/g fenoxycarb and that 90% reduction of population of larvae of German cockroach Blatella germanica is achieved after 3 weeks by exposing them to 100 ng/cm² fenoxycarb. In an attempt to improve this efficiency towards specific insects, pesticide combinations comprising two or more such active compounds have been tested and are also well known. In particular, EP-B-161,019 mentioned supra discloses admixing certain specific halobenzoylureas with other insecticides, especially organo-phosphates and pyrethroids. Research has also been directed to investigating the effects from exposure of a pest to the combination of a chitin synthesis inhibitor and of one or more juvenoids. In particular, M. H. Ross et al. in journal of Economic Entomology (1990) 83:2295-2305 and in Entomol.Exp.appl.(1991) 61:117-122 disclose the effects on mortality and sterility of German cockroach (Blatella germanica) nymphs achieved by combinations of fenoxycarb (a juvenoid) and diflubenzuron (a chitin synthesis inhibitor). It shows that the lowest concentrations of such combinations of IGRs at which exposure (one test per concentration) of large nymphs completely eliminated productive matings are concentrations of 2.0 ng/cm² fenoxycarb admixed with 100 ng/cm² diflubenzuron with respect to females (mortality 73%) and concentrations of 6.0 ng/cm² fenoxycarb admixed with 300 ng/cm² diflubenzuron with respect to males (mortality 47%), i.e. in each case a weight ratio diflubenzuron:fenoxycarb of 50:1. The authors of this study conclude that the enhanced effectiveness of diflubenzuron achieved by means of its combination with fenoxycarb express the effects expected from exposure to both a juvenoid and a chitin synthesis inhibitor.

Because they only affect hormonal systems unique to insects, IGRs are considered to be environmentally acceptable and are gaining acceptance as tools in cockroach pest management. Although the above-mentioned compounds and compositions may be effective against some insects, there is a continuing need for compositions which are either more efficient against a broader spectrum of pests or require lower amounts of active compounds to provide the same level of effectiveness as a matter of environmental concern and cost. In particular there is a need for compositions which may be effective against a wide range of insects. More specifically, there is a need for combinations of a chitin synthesis inhibitor and a juvenoid wherein the proportion of the chitin synthesis inhibitor can be reduced in comparison with the known combinations, i.e. wherein the ratio of the chitin synthesis inhibitor to the juvenoid and/or the combined amounts of both pesticides are as low as possible.

### SUMMARY OF THE INVENTION

The present invention is based on the unexpected discovery that the above-mentioned goals and other benefits can be achieved by suitable combinations of a specific class of juvenoids and a specific class of chitin synthesis inhibitors. In particular, it has now been found that, within certain broad limits of composition (i.e. in certain respective proportions or amounts of the active ingredients) easily determinable by those skilled in the art, the combination of a substituted phenoxyethylcarbamic acid ester, thiophenoxyethylcarbamic acid ester or phenoxythioethylcarbamic acid ester and an optionally substituted 4-(haloalkyl)phenoxyphenylurea as active ingredients, more specifically a combination of flufenoxuron and fenoxycarb, is able to provide a synergistic effect on the control of invertebrates, i.e. a synergistic protecting effect against pests, especially insects and acari. The present invention also provides agronomic and therapeutic compositions comprising the said combination of active ingredients together with an agronomically or therapeutically acceptable carrier. Finally the present invention also provides a method of protecting any living or non-living material, such as plants, fruits, seeds, building parts made of wood, thatch or the like, biodegradable materials and textiles against deterioration due to the action of pests such as insects. In the case of protecting building parts or textiles, the present invention thus provides protective compositions in the form of non-toxic paints and coatings such as varnishes, enamels and the like. Finally the present invention also provides the use of said combination of active ingredients for the control of invertebrates, in particular insects, and for the manufacture of an insecticide for the treatment of warm-blooded animals.

### DETAILED DESCRIPTION OF THE INVENTION

A first embodiment of the present invention relates to a composition comprising:

a) one or more carbamic acid derivative of the formula

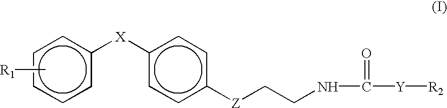

wherein

R₁ is hydrogen, fluorine, chlorine, C₁₋₆alkyl, trifluoromethyl, hydroxy or methoxy;

X is oxygen, carbonyl, methylene, sulphur or sulphonyl; Z is oxygen, methylene or sulphur;

Y is oxygen or sulphur and R₂ is C₁₋₆alkyl; and

b) one or more 4-(haloalkyl)phenoxyphenylurea of the formula

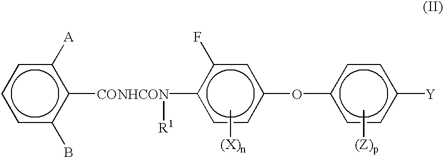

wherein

each of A, B, X and Z independently is fluorine or chlorine;

n is 0, 1, 2 or 3;

p is 0 or 1;

Y is a C₁₋₆haloalkyl group;

and R¹ hydrogen or —SNR²R³ wherein R² is C₁₋₆alkyl and R³ is C₁₋₆alkyl or —COR⁴ or —COOR⁴ wherein R⁴ is C₁₋₆alkyl, or R² and R³ together represent an alkylene group having 4 or 5 carbon atoms and optionally substituted by an alkoxycarbonyl group of up to 6 carbon atoms in the alkyl moiety,

in respective proportions such as to provide a synergistic effect against insects and acari.

Carbamic acid derivatives of the formula (I) are disclosed in U.S. Pat. No. 4,215,139. They include substituted phenoxyethylcarbamic acid esters, thiophenoxyethylcarbamic acid esters and phenoxythioethylcarbamic acid esters as can be seen from the list of preferred compounds at col.3 line 45 to col.4 line 20 of this document. A most preferred compound within such carbamic acid derivatives of the formula (I) is known as fenoxycarb, otherwise named ethyl 2-(4-phenoxyphenoxy)ethylcarbamate. 4-(Haloalkyl)phenoxyphenylureas of the formula (II) are disclosed in EP-B-0,161,019. A list of preferred such compounds is stated at col. 2 line 61 to col. 3 line 12 and in examples 5 to 23 of this document. A most preferred compound within such 4-(haloalkyl)phenoxyphenylureas is known as flufenoxuron, otherwise named 1-[4-(2-chloro-trifluoro-p-tolyloxy)-2-fluorophenyl]-3-(2,6-difluorobenzoyl)urea. Thus, a most preferred composition according to the present invention is a composition comprising fenoxycarb and flufenoxuron.

The term “C₁₋₆alkyl” as used herein, either by itself or as part of a group, unless otherwise stated, means straight and branched chain groups having 1 to 6 carbon atoms such as methyl, ethyl, propyl, isopropyl, butyl, isobutyl, sec-butyl, tert-butyl, pentyl, isopentyl, hexyl, isohexyl and the like. The term “alkoxy” as used herein, either by itself or as part of a group, unless otherwise stated, means methoxy, ethoxy, propoxy, iso-butoxy and the like. The term “halo” as used herein, either by itself or as part of a group, unless otherwise stated, means a halogen atom, preferably fluorine or chlorine. The term “C₁₋₆haloalkyl” as used herein means a C₁₋₆alkyl group substituted with one or more halogen atoms.

The relative proportions of compounds (a) and (b) in the embodied composition are those proportions which result in unexpected synergistic efficacy against pests, especially against insects and/or acari, when compared to a composition including, as an active ingredient, either compound (a) alone or compound (b) alone. As will readily be understood by those skilled in the art, the said synergistic efficacy may be obtained within various proportions of compounds (a) and (b) in the composition, depending on the kind of pest towards which efficacy is measured and the substrate to be treated. Based on the teachings of the present application, determination of the synergistic or possibly (for some proportions of compounds (a) and (b) applied to specific pests) non-synergistic efficacy of such compositions is well within the routine work of those skilled in the art. As a general rule, however, it may be said that for most pests the suitable proportions by weight of the amount of compound (a) to compound (b) in the active composition should lie in the range from 0.01:1 to 100:1. Preferably, this range is from 0.03:1 to 65:1. More preferably, this range is from 0.06:1 to 20:1. Most preferably, this range is from 0.12:1 to 8:1.

The active ingredients of the formulae (I) and (II) for use in the compositions according to the present invention should preferably present in a substantially pure form, i.e. free from chemical impurities (such as co-products or residual solvents) resulting from their manufacturing and/or handling processes in view to safely control the pest management programs for which they are intended. The active ingredients of the formulae (I) and (II) for use in the compositions according to the present invention may be present, when they possess at least an asymmetric carbon atom, either as a racemic mixture or in the form of a substantially pure stereoisomer or enantiomer of the said compound obtained from the racemic mixture by standard fractionation methods, including simulated moving bed technology. The term “substantially pure” as used hereinbefore means a purity (either chemical or optical), as determined by methods conventional in the art such as high performance liquid chromatography or optical methods, of at least about 96%, preferably at least 98% and more preferably at least 99%.

As is well known to those skilled in the art, most active ingredients of formula (I) are solid materials with melting points in the range of about 50° C. to about 140° C. and with very low solubility in water. In particular, fenoxycarb is very soluble in alcohols, ketones and toluene and is stable to hydrolysis in aqueous solutions at 50° C. Similarly, most active ingredients of formula (II) like flufenoxuron are solid materials with melting points in the range of about 65° C. to about 200° C., with very low solubility in water and with rather good solubility in halogenated hydrocarbons and ketones. These physical characteristics should be taken into account when formulating these active ingredients into agronomic, therapeutic or protective compositions, as explained hereinafter.

As previously mentioned, the compositions according to the present invention exhibit an unexpectedly improved pesticide activity and, more specifically, provide a synergistic protecting effect against pests such as insects. For this reason, the present invention also provides agronomic and therapeutic compositions comprising the said combination of active ingredients together with one or more agronomically or therapeutically acceptable carriers. The term “agronomically or therapeutically acceptable carrier” as used herein means any material or substance with which the composition of active ingredients (a) and (b) is formulated in order to facilitate its application/dissemination to the locus to be treated, for instance by dissolving, dispersing or diffusing the said composition, and/or to facilitate its storage, transport or handling without impairing its pesticidal effectiveness. The agronomically or therapeutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Preferably the agronomic and therapeutic compositions of the invention should contain from about 0.01 to 95% by weight of the combination of active ingredients (a) and (b). More preferably this range is from 0.1 to 90% by weight. Most preferably this range is from 1 to 80% by weight, depending on the type of formulation to be selected for specific application purposes, as further explained in details hereinafter.

The combination of active ingredients of formulae (I) and (II) is thus suitably used together with carriers and additives, including wetting agents, dispersing agents, stickers, adhesives, emulsifying agents and the like such as those conventionally employed in the art of formulation and consistent with agricultural and veterinary practices, i.e. carriers and additives which do not create permanent damage to soil and agronomic crops or to mammals. The agronomic and therapeutic compositions of the present invention may be prepared in any known manner, for instance by homogeneously mixing, coating and/or grinding the combination of active ingredients, in a one-step or multi-steps procedure, with the selected carrier material and, where appropriate, the other additives such as surface-active agents. The agronomic and therapeutic compositions of the present invention may also be prepared by micronisation, for instance in view to obtain them in the form of microspheres usually having a diameter of about 1 to 10 μm, namely for the manufacture of microcapsules for controlled or sustained release of the combination of active ingredients.

Examples of inert carrier materials suitable for use as solid carriers in the present invention, e.g. for dust concentrates and granular formulations, include natural and synthetic mineral fillers, for instance magnesium silicates such as talc; silica such as diatomaceous earth; aluminium silicate such as kaolinite, montmorillonite or mica; magnesium aluminium silicate such as attapulgite and vermiculite; calcium carbonate and calcium sulphate; carbon such as charcoal; sulphur; and highly dispersed silicic acid polymers. Suitable granulated absorbent carrier materials may be porous, for example pumice, broken brick, sepiolite or bentonite. In addition, a great number of pre-granulated materials or inorganic or organic nature can be used, e.g. especially dolomite or pulverised plant residues. Other inert carrier materials suitable for use as organic solid carriers include natural and synthetic resins (whether crude or formulated), for example organic waste polymeric products such as polyvinyl chloride, polyethylene, polypropylene, polyacrylates such as polymethylmethacrylate, polystyrene and mixed polymerisates thereof.

Suitable surface-active agents to be used in the agronomic and therapeutic compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenolpolyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, preferably halides, having 4 hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents which are conventional and suitable for this purpose may be found for instance in the following publications: “McCutcheon's Detergents and Emulsifiers Annual” (MC Publishing Crop., Ridgewood, N.J., 1981); “Tensid-Taschenbuch”, 2^{nd} ed. (Hanser Verlag, Vienna, 1981) and “Encyclopaedia of Surfactants (Chemical Publishing Co., New York, 1980-1981).

The agronomic and therapeutic compositions according to the present invention can be prepared in a number of ways, depending on the type of formulation selected. First, they can be prepared as dust concentrates, wherein the combination of active ingredients (a) and (b) constitutes about 20 to 80% of the said composition, which are normally extended (diluted) in the locus to be treated (e.g. a field) with further solid mineral or organic carrier so as to provide an active ingredient content of about 0.1 to 20% (preferably 0.5 to 10%) by weight for ultimate application. Alternatively wettable powder formulations, including the so-called “dry flowable powders”, can be made by incorporating the combination of active ingredients (a) and (b) into an inert finely divided solid carrier along with at least one surfactant such as described herein-before, in which case surfactant(s) amount to about 0.5 to 10% by weight of the said formulation. Water-dispersible granular products may be prepared by granulating, impregnating or agglomerating a suitable wettable powder formulation in order to obtain a granule average size between about 0.1 and 2.0 mm.

Alternatively, an emulsifiable concentrate formulation of the composition according to the present invention can also be obtained upon dilution of the combination of active ingredients (a) and (b) with at least an agronomically acceptable organic solvent (i.e. a liquid carrier) followed by the addition of at least a solvent-soluble emulsifying agent. Solvents suitable for this type of formulation are usually water-immiscible and belong to the hydrocarbon, chlorinated hydrocarbon, ketone, ester, alcohol and amide classes of solvents, and they can be properly selected by those skilled in the art based on the solubilities of compounds (a) and (b) respectively. Emulsifiable concentrates usually contain, in addition to the organic solvent(s), from about 10 to 50% by weight of the combination of active ingredients, from about 2 to 20% of emulsifying agent(s) and up to 20% other additives such as stabilisers, corrosion inhibitors and the like. The combination of active ingredients (a) and (b) may also be formulated as a suspension concentrate, which is a stable suspension of the active ingredients in a (preferably organic) liquid intended to be diluted with water before use. In order to obtain such a non-sedimenting flowable product, it is usually necessary to incorporate therein up to about 10% by weight of at least a suspending agent selected from known protective colloids and thixotropic agents. Other liquid formulations like aqueous dispersions and emulsions, for example obtained by diluting a wettable powder or a concentrate (such as previously described) with water, and which may be of the water-in-oil or the oil-in-water type, also lie within the scope of the present invention.

The present invention also provides protective compositions, for instance in the form of non-toxic paints, coatings or varnishes, comprising the said combination of active ingredients (a) and (b) together with one or more additives suitable for their formulation. Such additives are quite conventional in the art and include, for instance, at least an organic binder (preferably in aqueous form) such as an acrylic or vinyl-based emulsion; mineral carriers such as calcium carbonate; surface-active agents such as previously described with respect to the formulation of agronomic compositions; viscosity regulators; corrosion inhibitors; pigments such as titanium dioxide; stabilisers such as sodium benzoate, sodium hexametaphosphate and sodium nitrite; mineral or organic colorants and the like. The ways of formulating such additives together with active pesticidal ingredients such as those of the present invention is also well within the knowledge of those skilled in the art. Such protective compositions may be used not only to cure and/or limit the damaging effects of pests but also in order to prevent deterioration to occur on non-living material such as wood, textiles or other biodegradable materials which may be subjected to the harmful environment and effects of pests.

Other appropriate additives for use in the agronomic, therapeutic and protective compositions of the present invention may be solid or liquid and are suitable substances known in the art for preparing formulations for treating plants or plant products, in particular wood, as well as for treating building parts and construction materials, textiles (namely through impregnation or surface treatment) and mammals, while providing a further protective effect namely for storing and handling purposes. Such additives may comprise, for example, antioxidants, UV-absorbers, stabilisers, odour masking agents, viscosity enhancers and the like.

The agronomic, therapeutic and protective compositions according to the present invention can be applied by a number of conventional methods for pesticides, such as hydraulic spray, air-blast spray, aerial spray, atomising, dusting, scattering or pouring. The most appropriate method will be chosen by those skilled in the art in accordance with the intended objectives and the prevailing circumstances, namely the kind of pests (in particular insects) to be controlled, the type of equipment available and the type of living or non-living material to be protected.

The agronomic and therapeutic compositions according to the present invention can also be mixed with other agro-chemically active materials such as fertilisers or fertilising materials before their application. For instance particles of fertilisers or fertilising ingredients, such as ammonium sulphate, ammonium nitrate, ammonium phosphate or mixtures thereof, can be coated with a composition of the invention, using coating techniques well known in the art of fertilisation. Solid compositions according to the present invention and solid fertilising materials can also be admixed and/or granulated together while using conventional blending or granulating equipment. This will result in fertilising compositions, commonly comprising from about 1 to 25% by weight of the said agronomic composition, which promote the rapid growth of desired plants and at the same time protect the said plants against the harmful effects of pests such as insects. Other agro-chemically active materials suitable for admixture with the compositions of the invention include fungicides such as dithiocarbamates, nitrophenol derivatives, heterocyclic compounds (including thiophtalimides, imidazoles, triazines, thiadiazoles, triazoles and the like), acylalanines, phenylbenzamides and tin compounds; herbicides such as trichloroacetic and aromatic carboxylic acids and their salts, substituted ureas and triazines, diphenyl ether derivatives, anilides, uraciles and nitriles; and insecticides, other than those belonging to formulae (I) and (II), being able of either interfering with the hormonal system or of acting as chitin synthesis inhibitors.

If deemed necessary for some type of application or treatment, the agronomic and therapeutic compositions according to the present invention can be formulated as controlled-release or sustained-release compositions while using methods consistent with conventional agronomic and therapeutic (both pharmaceutical and veterinary) practices. Additional ingredients may thus be included in order to control the duration of action of the active ingredients (a) and (b) in the said compositions. Controlled-release compositions may thus be achieved by selecting appropriate polymer carriers such as for example polyesters, poly-aminoacids, polyvinyl pyrrolidone, ethylene-vinyl acetate copolymers, methylcellulose, carboxymethylcellulose, protamine sulfate and the like. The rate of release of active ingredients and their duration of action may also be controlled by incorporating them into particles, e.g. microcapsules, of a polymeric substance such as hydrogels, polylactic acid, hydroxymethylcellulose, polymethyl methacrylate and the other above-described polymers. Such methods include colloid active ingredient delivery systems such as liposomes, microspheres, microemulsions, nanoparticles, nanocapsules and so on.

The active ingredient compositions—i.e. mixtures of compounds (a) and (b)—of the present invention are particularly suitable for the control of invertebrates, more particularly of insects. Examples of representative insects against which the compositions of the present invention are useful include these belonging to the orders (named after the commonly admitted classification) Isoptera (e.g. termites), Diptera (flies, especially these belonging to the families Cecidomyiidae, Syrphidae (such as hover flies which are predaceous of cotton), Tachinidae and Agromyzidae), Lepidoptera (butterfies and moths, especially these belonging to the families Pyralidae (stem borers), Arctiidae (tiger moths and wooly bear caterpillars), Noctuidae such as cutworms and armyworms), Homoptera (leafhoppers and aphids, especially these belonging to the families Cicadellidae and Aphididae), Hymenoptera (bees, wasps and ants, especially these belonging to the families Braconidae, Cephidae (stem sawflies), Eurytomidae (jointworms), Formicidae, Ichneumonidae, Tiphiidae, Trichogrammatidae and Vespidae such as hornets), Coleoptera (beetles, especially these belonging to the families Scarabaeidae, Elateridae, Curculionidae (weevils) and Tenebronidae), Orthoptera (grasshopers, especially these belonging to the families Acrididae, Tettigoniidae (crickets) and Gryllidae, and blattids belonging to the family Blattidae like cockroach), Hemiptera (bugs, especially these belonging to the families Miridae, Nabidae, Lygaeidae, Anthocoridae, Reduviidae and Pentatomidae), Psocoptera, Thysanoptera (thrips, especially these belonging to the family Thripidae), Neuroptera (lace-wings, especially these belonging to the families Chrysopidae and Hemerobiidae) and Dermatophagoides spp. (housedust mite). A more detailed description of these insects and their parasitic action may be found in the following publication: “Introduction to Insect pest management” (John Wiley & Sons).

The agronomic, therapeutic and protective compositions of the present invention may also be useful against some arthropods other than insects, such as Arachnida, particularly these belonging to the order Acarina such as namely acari, harvest-mites, Sarcoptidae (including itch-mites, cheese-mites, Tyrolichus casei and the like) and Trombidiae. Among acari, special attention may be paid to ticks and mites. Mites are very small arthropods (1-3 mm long) that feed on plants, animals and organic debris. One important plant-feeding mite is the spider mite (Tetranychus urticae) which injures many crops in dry climates or during droughts in wetter regions. Chiggers (larvae of Trombicula alfreddugesi) are another kind of mites which infest humans, causing intense itching when they inject enzymes to dissolve skin tissue on which they feed. Mange mites (Sarcoptes scabiei) feed in the skin of many animals, including hogs, horses, dogs and humans. In humans, these mites cause scabies, a skin condition often noted in the elderly. Ticks are acarids larger than mites (usually 10-20 mm long) with leathery integument, which feed only on animals (mammals, birds and reptiles) by sucking blood from and sometimes transmitting disease-causing organisms to their hosts. Examples of important tick pests include the lone star tick (Amblyomma americanum) and the dog tick (Dermacentor variabilis) which attack humans, dogs and livestock and are transmitters of the causal agent of Rocky Mountain spotted fever. Other examples of harmful ticks and mites include Gossypii, Boophilus, Anocentor, Haemaphysalis, Hyalomma, Ixodes, Rhipicentor, Margaropus, Rhipicephalus, Argas and Latus.

It should be understood that the above enumeration of invertebrates, in particular insects and acari, affected by the compositions of the invention is provided for illustrative purposes only and not with the aim of limiting the scope of the present invention. The active ingredient compositions—i.e. mixtures of compounds (a) and (b)—of the present invention are particularly suitable for the control of the grain weevil (Sitophilus granarius) and termites (order Isoptera). Within termites, special attention will be paid to species commonly known as Zootermapsis (present on the Pacific coast of North America), Reticulitermites lucifugus and Reticulitermites flavipes (respectively present on the Atlantic coast of North America and on the Atlantic coast of continental Western Europe) and Reticulitermites speratus (present in Far East Asia).

It is estimated that about 3,500 insect species fall into the pest category, causing injury to crop plants, forests and ornamentals, annoyance, injury and death to humans and domesticated animals, and destruction or value depreciation of stored products and possessions. Insects injure plants by feeding on them, consuming plant tissues with various types of chewing mouthparts and removing plant juices with piercing-sucking mouthparts. Some insects, e.g. aphids and leafhoppers, also transmit various kinds of plant pathogens, including bacteria, fungi, viruses and mycoplasmas that subsequently cause losses from diseases. Other insects, e.g. the potato leafhopper Empoasca fabae, inject toxins that influence the plant's physiology and subsequently result in yield and quality decrease. Examples of direct injury to plants include that of the boll weevil Anthonomus grandis (which destroys cotton squares), the codling moth Cydia pomonella (which feeds inside apples) and the bark beetle Scolytus (which bore into trees). The agronomic compositions according to the present invention are able to help in curing the above stated problems by the fact that they possess advantageous curative and preventive pesticide activity in order to protect plants, in particular culture plants (like vegetables) and ornamental plants. They can therefore be used to protect such plants or parts of such plants, e.g. fruits, blossoms, flowers, foliage, stems, roots, tubers which might be infected, harmed or destroyed by pests such as insects and arachnids, whereby later-growing parts of such plants will be protected against such pests. They can further be used as a preventive protection means for instance in seed disinfection (namely for cereal grains).

As a general consideration, the agronomic, therapeutic and protective compositions of the present invention are particularly attractive due to their good plant and animal tolerance and lack of environmental problems when used according to the recommended dosages. In particular, flufenoxuron has no recorded phyto-toxicity and both flufenoxuron and fenoxycarb are rapidly degraded in soil. Moreover the high efficiency of these compositions, even at very low dosages, attributable to the unexpected synergistic effect between flufenoxuron and fenoxycarb, further reduces any possibly undesired effects.

As examples of the wide variety of culture plants in which the combination of active ingredients according to the present invention can be used, there may be named for example cereals (e.g. wheat, barley, rye, oats, rice, sorghum and the like), beets (e.g. sugar beet and fodder beet), pomes, stone fruits and berries (e.g. apples, pears, plums, peaches, almonds, cherries, strawberries, raspberries and blackberries), leguminous plants (e.g. beans, lentils, peas and soy beans), oleaginous plants (e.g. rape, mustard, poppy, olive, sunflower, coconut, castor-oil plant, cocoa and ground-nuts), cucurbitaceae (e.g. pumpkins, gherkins, melons, cucumbers and squashes), fibrous plants (e.g. cotton, flax, hemp and jute), citrus fruits (e.g. orange, lemon, pomelo and mandarin), vegetables (e.g. spinach, lettuce, asparagus, cabbages and turnips, carrots, onions, tomatoes, potatoes, hot and sweet peppers) laurel-like plants (e.g. avocado, cinnamon, camphor tree) and other plants such as maize, tobacco, nuts, coffee, sugar-cane, tea, vines, hops, bananas and rubber plants, as well as ornamental trees. This enumeration is provided for illustrative purposes only and not with the aim of limiting the scope of the present invention.

The protective compositions of the present invention are useful for the protection of buildings and building parts, during and/or after their construction, and for the protection of construction and biodegradable materials against insects of the order Isoptera, in particular against termites (e.g. Reticulitermes flavipes) and other wood-destroying insects. They may also be used to combat insects damaging to textiles such as moths (e.g. Tineola biselliella) and the like. Also included within the scope of this invention is the protection of articles made from biodegradable material such as agglomerated wood used in the manufacture of palettes for storing and packing products of any kind and which quite often remain in the outside. Another use of the invention is for the protection of food products (such as rice, cereals, sugar and the like) during their storage before consumption.

The present invention therefore also provides a method for protecting any living or non-living material, such as plants, fruits, seeds, food, buildings and building parts (especially when made of wood, thatch or the like), biodegradable materials and textiles against deterioration due to the action of pests such as insects.

Those skilled in the art of building protection, during and/or after construction, are familiar with pesticide application methods suitable for this purpose. Briefly, this can be done by creating chemical and/or physical barriers by means of bait boxes, by pouring, spraying or injecting the pesticide composition of the invention in effective and lasting doses, by impregnating or painting or coating the surface(s) of the building part to be protected.

It is well known that bloodsucking ectoparasites of the orders Insecta and Acarina can infest or attack many useful warmblooded animals, especially mammals, gallinaceae and anatidae, including farm animals such as cattle, swine, sheep, goats, poultry (chickens, turkeys, geese and the like), bearing animals (minks, chinchillas, rabbits and the like) and pet animals such as dogs and cats. Examples of such ectoparasites include Ctenocephalides felis and Ctenocephalides canis (cat and dog fleas), as well as lice, mosquitos, tabanids, tsetse and other biting flies, and Acarina such as Ixodes (ticks) and the like. Apart from the unpleasant result for these animals to be bitten or sucked, it is also well known that many of these ectoparasites have the ability to transmit serious diseases to animals and also sometimes, either directly or through physical contact between animals and human beings, to humans. With respect to farm animals, diseases induced by these ectoparasites often result in mortality involving a reduction of the productivity in stock-farming. With respect to humans they also result in health problems, in particular for persons that are hypersensitive to the venoms injected when insects bite or sting, which need to be solved. Among the most important diseases transmitted by insects bites are malaria, yellow fever, filariasis and several types of encephalitis. A prevention of the occurrence of such problems involved by the presence of these ectoparasites in animals and humans is therefore of primary importance for both economic and health reasons and may be achieved by the therapeutic compositions of the present invention.

Except for lice, the above-mentioned ectoparasites spend a major portion of their life cycle off the host in its environment. Control measures are therefore to interfere with controlling the parasites, directly or indirectly via their off-spring, on the host (via topical application on at least part of the skin) and off the host. The latter relates to combating free living stages of the parasite life cycles in its environment. Therefore the present invention provides the use of a composition of active ingredients (a) and (b) or a therapeutic formulation resulting therefrom for the manufacture of an insecticide for the preventive and curative treatment of warmblooded animals, in particular mammals, gallinaceae and anatidae. The present invention also provides a method for treating warmblooded animals against the harmful effects of insects and/or acari, comprising the administration or application to the said warmblooded animals of a therapeutically effective amount of a composition of active ingredients (a) and (b) or a therapeutic formulation resulting therefrom, such as previously disclosed. The compositions of the present invention may, by means of their topic administration to an animal or human host, prevent ectoparasite infestations namely by-reducing the fertility of any new ectoparasite and/or by preventing them to become adults. Since, at the dosages recommended for such topic administration, active compounds (a) and (b) such as flufenoxuron and fenoxycarb do not result in hazards to vertebrates like animals and humans, the compositions of this invention can be used safely for treating such animals, especially mammals and humans. With respect to animals, topic administration can take the form of, but is not restricted to, any device providing a controlled release (preferably a sustained or slow release) of the pesticide composition such as ear tags, ear tape and collars, or another liquid or flowable formulation such as shampoos, creams, ointments and the like.

As previously indicated, the combination of active ingredients of formulae (I) and (II) is preferably applied in the form of compositions wherein both said ingredients are intimately admixed in order to ensure simultaneous administration to plants or animals or simultaneous application to biodegradable materials, building parts and textiles to be protected. Administration or application of both active ingredients of formulae (I) and (II) can also be a “sequential-combined” administration or application, i.e. compound (a) and compound (b) are administered or applied alternatively or sequentially in the same place in such a way that they will necessarily become admixed together at the locus to be treated. This will be achieved namely if sequential administration or application takes place within a short period of time e.g. within less than 24 hours, preferably less than 12 hours. This alternative method can be carried out for instance by using a suitable single package comprising at least one container filled with a formulation comprising the active compound (a) and at least one container filled with a formulation comprising the active compound (b). Therefore the present invention also encompasses a product containing:

a composition comprising (a) one or more carbamic acid derivative of the formula (I), and

a composition comprising (b) one or more 4-(haloalkyl)phenoxyphenylurea of the formula (II) as a combination for simultaneous or sequential use, wherein compounds (a) and (b) are in respective proportions such as to provide a synergistic effect against insects and/or acari.

A particular mode of administration or application of the agronomic compositions of the present invention is by application thereof to the aboveground parts of plants, in particular to their leaves (leaf-application). The frequency of application and the recommended dosage will be selected in accordance with the biological and climatic conditions of life of the causative agent (pest). The compositions of the present invention can also be applied directly to the soil and then get into the plants through the root system (systemic activity), if the locus of the plants is either sprayed with a liquid composition or if a solid formulation, e.g. in the form of a granulate, is added to the soil (soil application). The compositions of the present invention can also be conveniently administered by coating on seeds. In most cases, and especially when the compositions of the present invention are to be used directly onto plant products like seeds, e.g. wheat grains, the concentration of compound (b)—e.g. flufenoxuron—is preferably in the range of about 0.5 to 300 mg/l (mg per liter of test mixture) and the concentration of compound (a)—e.g. fenoxycarb—is preferably in the range of about 0.125 to 50 mg/l.

The agronomic compositions of the present invention are particularly useful in post-harvest treatment of fruits, especially citrus fruits. In the latter instance, the fruits will conveniently be sprayed with or dipped or drenched into a liquid formulation or the fruits may be coated with a waxy composition comprising the combination of both active ingredients. Such a waxy composition may conveniently be prepared by thoroughly mixing a suspension concentrate of the said active ingredients with a suitable wax.

The combinations of active ingredients of formulae (I) and (II) and the agronomic, therapeutic and protective compositions resulting therefrom may further comprise at least another biologically active ingredient, e.g. selected from insecticides, pesticides, herbicides, plant growth regulators, fertilisers, antimicrobial agents (in particular fungicides and bactericides), admissible for use in plants, animals and humans, building parts and materials, textiles and other biological material needing protection.

Antimicrobial agents which may be used in combination with active substances (a) and (b) include halogenated phenols, chlorinated diphenylethers, aldehydes, alcohols such as phenoxyethanol, carboxylic acids and their derivatives, organometallic compounds such as tributyltin compounds, iodine compounds, mono- and polyamines, sulfonium and phosphonium compounds; mercapto compounds as well as their alkaline, alkaline-earth and heavy metal salts; ureas such as trihalocarbanilide, isothia- and benzisothiazolone derivatives. Insecticides which may be used in the compositions according to the present invention include natural ones, e.g. nicotine, rotenone, pyrethrum and the like, and synthetic ones like chlorinated hydrocarbons, organophosphorus compounds, biological insecticides (e.g. products derived from Bacillus thuringiensis), synthetic pyrethroids, organosilicon compounds, nitro-imines and nitromethylenes. Herbicides and plant growth regulators which may be used in the compositions according to the present invention are also well known to those skilled in the art.

The following examples are provided in order to illustrate the invention, not to limit its scope in any of its aspects. All percentages, unless otherwise stated, are expressed by weight.

### EXAMPLES A TO J (COMPARATIVE) AND 1 TO 24

The technical grade of flufenoxuron (purity 96.6%) used in all experiments was supplied by American Cyanamid under the trade name Cascade®. The technical grade of fenoxycarb (purity 97%) used in all experiments was supplied by Ciba Geigy® under the trade name Insegar®. Each compound was applied at five different concentrations. Ethanol solutions containing 8, 4, 2, 1 or 0.5 ppm (w/v) of flufenoxuron respectively or 2, 1, 0.5, 0.25 or 0.125 ppm (w/v) of fenoxycarb were prepared. These concentrations (in mg/l or ppm) of flufenoxuron and fenoxycarb were combined in all possible ways as indicated in the table below.

Treatment of wheat grains was executed in 24-well culture plates. One ml of the ethanol solution to be tested was added to 1 gram of grains. The test plates were then placed on a hot plate at 60° C. for a period of 4 hours in order to allow ethanol to evaporate. Fifty adult beetles (Sitophilus granarius) from a two-weeks old, mixed population (25 males, 25 females) were then added to each sample. The plates were then incubated at 30° C., with a relative humidity of 60% and under a day:night regime of 16:8.

After 11 days, the adult beetles which by then had deposited eggs into the kernels, were separated from the grain by using a sieve. Seven weeks after addition of the adult beetles to the wheat grains, evaluation of the test was performed by counting the number of beetle progeny produced. Measured activity of the compounds, alone or in combination, was expressed in % reduction of the treated versus untreated population, respectively. Results presented in the table below are the avrage of 6 replicates.

The table below also provides the expected activity of each combination of ingredients, as may be calculated using the well-known Limple's formula such as disclosed for instance by Richter D. L., Pestic.Sci. (1987) 16: 309-315):

E_{c}=X+Y−[(X.Y)/100]

where E_{c} is the expected additive response, X is the observed percentage control when one compound is applied alone and Y is the observed percentage control when the other compound is applied alone.

TABLE

| | | | Measured | Calculated |
|---|---|---|---|---|
| | Flufenoxuron | Fenoxycarb | activity | activity |
| Example | conc. (ppm) | conc. (ppm) | (%) | (%) |
| A | 8 | — | 80 | — |
| B | 4 | — | 60 | — |
| C | 2 | — | 20 | — |
| D | 1 | — | 0 | — |
| E | 0.5 | — | 0 | — |
| F | — | 2 | 90 | — |
| G | — | 1 | 80 | — |
| H | — | 0.5 | 60 | — |
| I | — | 0.25 | 40 | — |
| J | — | 0.125 | 20 | — |
| 1 | 8 | 2 | 100 | 98 |
| 2 | 8 | 1 | 100 | 96 |
| 3 | 8 | 0.5 | 99 | 92 |
| 4 | 8 | 0.25 | 99 | 88 |
| 5 | 8 | 0.125 | 100 | 84 |
| 6 | 4 | 2 | 100 | 96 |
| 7 | 4 | 1 | 100 | 92 |
| 8 | 4 | 0.5 | 99 | 84 |
| 9 | 4 | 0.25 | 99 | 76 |
| 10 | 4 | 0.125 | 96 | 68 |
| 11 | 2 | 2 | 100 | 92 |
| 12 | 2 | 1 | 98 | 84 |
| 13 | 2 | 0.5 | 93 | 68 |
| 14 | 2 | 0.25 | 82 | 52 |
| 15 | 2 | 0.125 | 87 | 36 |
| 16 | 1 | 2 | 99 | 90 |
| 17 | 1 | 1 | 95 | 80 |
| 18 | 1 | 0.5 | 92 | 60 |
| 19 | 1 | 0.25 | 65 | 40 |
| 20 | 1 | 0.125 | 32 | 20 |
| 21 | 0.5 | 2 | 99 | 90 |
| 22 | 0.5 | 1 | 97 | 80 |
| 23 | 0.5 | 0.5 | 70 | 60 |
| 24 | 0.5 | 0.25 | 55 | 40 |

Synergy resulting from the combined use of both compounds is clearly demonstrated to occur when the experimentally observed effect is greater than the corresponding E_{c} value, i.e. in each of the above operative examples 1 to 24.

1. A composition comprising:
a) one or more carbamic acid derivative of the formula 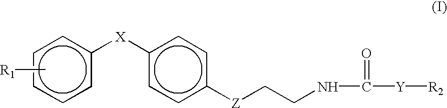
wherein
R₁ is hydrogen, fluorine, chlorine, C₁₋₆alkyl, trifluoromethyl, hydroxy or methoxy; X is oxygen, carbonyl, methylene, sulphur or sulphonyl; Z is oxygen, methylene or sulphur; Y is oxygen or sulphur and R₂ is C₁₋₆alkyl; and
b) one or more 4-(haloalkyl)phenoxyphenylurea of the formula 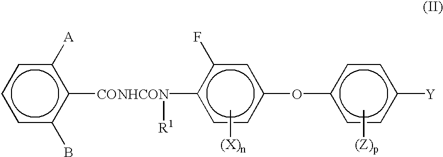
wherein
each of A, B, X and Z independently is fluorine or chlorine;
n is 0,1,2 or 3;
p is 0 or 1;
Y is a C₁₋₆haloalkyl group;
and R¹ is hydrogen or —SNR²R³ wherein R² is C₁₋₆alkyl and R³ is C₁₋₆alkyl or —COR⁴ or —COOR⁴ wherein R⁴ is C₁₋₆alkyl, or R² and R³ together represent an alkylene group having 4 or 5 carbon atoms and optionally substituted by an alkoxycarbonyl group of up to 6 carbon atoms in the alkyl moiety,
in respective proportions such as to provide a synergistic effect against insects and/or acari.

2. A composition according to claim 1, wherein the carbamic acid derivative of formula (I) is fenoxycarb.

3. A composition according claim 1 or claim 2, wherein the 4-(haloalkyl)phenoxyphenylurea of formula (II) is flufenoxuron.

4. A composition according to any of claims 1 to 3, wherein the proportions by weight of the amount of compound (a) to compound (b) in the active composition are in the range from 0.01:1 to 100:1.

5. An agronomic or therapeutic composition comprising a composition of active ingredients according to any of claims 1 to 4 and further comprising at least an agronomically or therapeutically acceptable carrier.

6. An agronomic or therapeutic composition according to claim 5, wherein the agronomically or therapeutically acceptable carrier is an inert solid carrier suitable for dust concentrate or granular formulations.

7. An agronomic or therapeutic composition according to claim 5 or claim 6, further comprising at least one surface-active agent selected from anionic surfactants, non-ionic surfactants and cationic surfactants.

8. An agronomic or therapeutic composition according to claim 7, in the form of a wettable powder formulation comprising an inert solid carrier and wherein surfactants amount to 0.5 to 10% by weight of the said formulation.

9. An agronomic or therapeutic composition according to claim 5, wherein the agronomically or therapeutically acceptable carrier is an inert liquid carrier suitable for emulsifiable concentrate formulations.

10. A protective composition comprising a composition of active ingredients according to any of claims 1 to 4 and further comprising at least an additive suitable for the formulation of paints, coatings or varnishes.

11. An agronomic, therapeutic or protective composition according to any of claims 5 to 10, further comprising at least a biologically active ingredient.

12. An agronomic, therapeutic or protective composition according to claim 11, wherein the biologically active ingredient is selected from herbicides, plant growth regulators, fertilisers and antimicrobial agents.

13. Use of a composition according to any of claims 1 to 12 for the control of insects and/or acari.

14. A method of protecting living or non-living material against insects and/or acari, wherein the said method comprises administration or application of a composition according to any of claims 1 to 12.

15. A method according to claim 14, wherein the living or non-living material to be protected is selected from plants, seeds, fruits, food, biodegradable materials, buildings and textiles.

16. A method according to claim 14 or claim 15, comprising the post-harvest treatment of fruits.

17. Use of a composition according to any of claims 1 to 12 for the manufacture of an insecticide or anti-acari composition for the treatment of warmblooded animals.

18. A product containing:
a composition comprising (a) one or more carbamic acid derivative of the formula 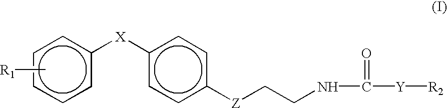
wherein
R₁ is hydrogen, fluorine, chlorine, C₁₋₆ alkyl, trifluoromethyl, hydroxy or methoxy; X is oxygen, carbonyl, methylene, sulphur or sulphonyl; Z is oxygen, methylene or sulphur; Y is oxygen or sulphur and R₂ is C₁₋₆ alkyl, and
a composition comprising (b) one or more 4-(haloalkyl)phenoxyphenylurea of the formula 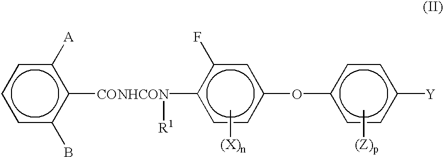
wherein
each of A, B, X and Z independently is fluorine or chlorine;
n is 0,1,2 or 3;
p is 0 or 1;
Y is a C₁₋₆ haloalkyl group; and R¹ is hydrogen or —SNR²R³ wherein R² is C₁₋₆ alkyl and R³ is C₁₋₆ alkyl or —COR⁴ or —COOR⁴ wherein R⁴ is C₁₋₆ alkyl, or R² and R³together represent an alkylene group having 4 or 5 carbon atoms and optionally substituted by an alkoxycarbonyl group of up to 6 carbon atoms in the alkyl moiety,
as a combination for simultaneous or sequential use, wherein compounds (a) and (b) are in respective proportions such as to provide a synergistic effect against insects and/or acari.
